SEQUENCE LISTING (4 sequences)

SEQ ID NO: 1 - protein, 4 aa, Artificial Sequence
  First 4 amino acids of B-domain of Factor VIII (FVIII)
SFSQ

SEQ ID NO: 2 - protein, 10 aa, Artificial Sequence
  Last 10 amino acids of B-domain of Factor VIII (FVIII)
NPPVLKRHQR

SEQ ID NO: 3 - protein, 1457 aa, Artificial Sequence
  B-Domain Deleted Human Factor VIII
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 4 - protein, 2351 aa, Homo sapiens [sequence omitted]

(19) United States
(12) Patent Application Publication
(10) Pub. No.: US 2014/0274902 A1
(43) Pub. Date: Sep. 18, 2014

(54) RECOMBINANT FVIII FORMULATIONS

(71) Applicant: BAYER HEALTHCARE, LLC, Whippany, NJ (US)

(72) Inventors: Deqian Wang, Concord, CA (US); Xinghang Ma, Dublin, CA (US); Nelly Tsvetkova, Vallejo, CA (US)

(73) Assignee: BAYER HEALTHCARE, LLC, Whippany, NJ (US)

(21) Appl. No.: 14/214,118

(22) Filed: Mar. 14, 2014

Related U.S. Application Data
(60) Provisional application No. 61/779,495, filed on Mar. 13, 2013.

(51) Int. Cl. A61K 47/48; A61K 38/37

(52) U.S. Cl. CPC A61K 47/48215; A61K 38/37
USPC 514/14.1

(57) ABSTRACT

Provided are liquid and lyophilized rFVIII formulations, including full-length rFVIII (FL-rFVIII) formulations, B-domain deleted rFVIII (BDD-rFVIII) formulations, and BDD-rFVIII mutant (BDD-rVIII mutant) formulations. Also provided are liquid and lyophilized PEGylated rFVIII (PEG-rFVIII) formulations, including PEGylated full-length rFVIII (PEG-FL-rFVIII) formulations, PEGylated B-domain deleted rFVIII (PEG-BDD-rFVIII) formulations, and PEGylated BDD-rFVIII mutant (PEG-BDD-rFVIII mutant) formulations.

### CROSS-REFERENCE TO RELATED APPLICATIONS

The present application is a continuation application of U.S. Application No. 61/799,495, entitled “RECOMBINANT FVIII FORMULATIONS” and filed Mar. 15, 2013, the entire disclosure of which is expressly incorporated herein by reference.

### SEQUENCE LISTING SUBMISSION

The present application includes a Sequence Listing in electronic format as a txt file titled “Sequence-Listing-17207-0008USU1” which was created on Mar. 12, 2014 and which has a size of 32.2 kilobytes (KB). The contents of txt file “Sequence-Listing-17207-0008USU1” are incorporated by reference herein.

### BACKGROUND

The present disclosure provides liquid and lyophilized rFVIII formulations, including full-length rFVIII (FL-rFVIII) formulations, B-domain deleted rFVIII (BDD-rFVIII) formulations, and BDD-rFVIII mutant (BDD-rVIII mutant) formulations. Also provided are liquid and lyophilized PEGylated rFVIII (PEG-rFVIII) formulations, including PEGylated full-length rFVIII (PEG-FL-rFVIII) formulations, PEGylated B-domain deleted rFVIII (PEG-BDD-rFVIII) formulations, and PEGylated BDD-rFVIII mutant (PEG-BDD-rFVIII mutant) formulations. As used herein, a BDD-rFVIII mutant comprises one or more cysteine substitutions within the amino acid sequence of BDD-rFVIII.

Hemophilia A is caused by deficiencies in coagulation Factor VIII (FVIII). The treatment involves intravenous injection of recombinant human FVIII (rFVIII). Due to the short circulating half-life of rFVIII, frequent injections are required. Injections are either administered on demand due to bleeding event, or as a prophylactic therapy administered several times a week. The need to frequent injections affects the patient's quality of life. PEGylation has been shown to increase the half-life of protein therapeutics. PEGylation is the covalent attachment of long-chain polyethylene glycol (PEG) molecules to proteins.

U.S. Pat. No. 5,763,401 discloses stable, albumin-free, lyophilized full-length recombinant FVIII (FL-rFVIII) formulations. U.S. Pat. No. 7,632,921 discloses FVIII mutants, including B-domain deleted FVIII (BDD-rFVIII), BDD-rFVIII mutants, and cysteine enhanced FVIII mutants that are covalently bound to one or more biocompatible polymers such as polyethylene glycol. Mei et al., Blood 116:270 (2010) discloses FVIII mutants with introduced surface-exposed cysteines to with a polyethylene glycol polymer was specifically conjugated. Each of these references is incorporated by reference herein in its entirety.

### SUMMARY

The present disclosure provides liquid and lyophilized rFVIII formulations, including full-length rFVIII (FL-rFVIII) formulations, B-domain deleted rFVIII (BDD-rFVIII) formulations, and BDD-rFVIII mutant (BDD-rVIII mutant) formulations. Also provided are liquid and lyophilized PEGylated rFVIII (PEG-rFVIII) formulations, including PEGylated full-length rFVIII (PEG-FL-rFVIII) formulations, PEGylated B-domain deleted rFVIII (PEG-BDD-rFVIII) formulations, and PEGylated BDD-rFVIII mutant (PEG-BDD-rFVIII mutant) formulations. As used herein, a BDD-rFVIII mutant comprises one or more cysteine substitution(s) within the amino acid sequence of BDD-rFVIII.

Within certain aspects, the present disclosure provides rFVIII formulations, including FL-rFVIII formulations, BDD-rFVIII formulations, and BDD-rFVIII-mutant formulations, as well as PEG-rFVIII formulations, including PEG-FL-rFVIII formulations, PEG-BDD-rFVIII formulations, and PEG BDD-rFVIII-mutant formulations, which rFVIII and PEG-rFVIII formulations comprise:
(a) from about 0 mM to about 20 mM or about 50 mM histidine;
(b) from about 0 mM to about 29 mM, or about 34 mM, or about 58 mM, or about 100 mM, or about 300 mM of a sugar or sugar alcohol;
(c) from about 1 mM to about 2 mM, to about 2.5 mM, to about 5 mM, to about 10 mM, to about 15 mM calcium chloride;
(d) from about 100 mM to about 150 mM, or about 200 mM, or about 220 mM, or about 250 mM sodium chloride;
(e) from about 20 ppm to about 50 ppm, or about 80 ppm, or about 100 ppm, or about 120 ppm, or about 200 ppm of a non-ionic surfactant; and
(f) from about 0.1 μg/ml to about 300 μg/ml, or about 1.0 μg/ml to about 200 μg/ml, or about 10 μg/ml to about 125 μg/ml of a rFVIII selected from a FL-rFVIII, a BDD-rFVIII, and a BDD-rFVIII-mutant or a PEG-rFVIII selected from a PEG-FL-rFVIII, a PEG-BDD-rFVIII, and a PEG BDD-rFVIII-mutant;
   wherein the rFVIII formulation has a pH from about pH 6.0 to about pH 6.5, or about pH 7.0, or about pH 7.5.

Within other aspects, the present disclosure provides rFVIII formulations, including FL-rFVIII formulations, BDD-rFVIII formulations, and BDD-rFVIII-mutant formulations, as well as PEG-rFVIII formulations, including PEG-FL-rFVIII formulations, PEG-BDD-rFVIII formulations, and PEG BDD-rFVIII-mutant formulations, which rFVIII and PEG-rFVIII formulations comprise:
(a) from about 0 mM to about 20 mM or about 50 mM MOPS;
(b) from about 0 mM to about 29 mM, or about 34 mM, or about 58 mM, or about 100 mM, or about 300 mM of a sugar or sugar alcohol;
(c) from about 1 mM to about 2 mM, to about 2.5 mM, to about 5 mM, to about 10 mM, to about 15 mM calcium chloride;
(d) from about 100 mM to about 150 mM, or about 200 mM, or about 220 mM, or about 250 mM sodium chloride;
(e) from about 20 ppm to about 50 ppm, or about 80 ppm, or about 100 ppm, or about 120 ppm, or about 200 ppm of a non-ionic surfactant; and
(f) from about 0.1 μg/ml to about 300 μg/ml, or about 1.0 μg/ml to about 200 μg/ml, or about 10 μg/ml to about 125 μg/ml of a rFVIII selected from a FL-rFVIII, a BDD-rFVIII, and a BDD-rFVIII-mutant or a PEG-rFVIII selected from a PEG-FL-rFVIII, a PEG-BDD-rFVIII, and a PEG BDD-rFVIII-mutant;
   wherein the rFVIII formulation has a pH from about pH 6.0 to about pH 6.5, or about pH 7.0, or about pH 7.5.

Within further aspects, the present disclosure provides rFVIII formulations, including FL-rFVIII formulations, as well as PEG-rFVIII formulations, including PEG-FL-rFVIII formulations, PEG-BDD-rFVIII formulations, and PEG BDD-rFVIII-mutant formulations, which rFVIII and PEG-rFVIII formulations comprise:
(a) from about 0 mM to about 20 mM or about 50 mM histidine;
(b) from about 0 mM to about 29 mM, or about 34 mM, or about 58 mM, or about 100 mM, or about 300 mM of a sugar or sugar alcohol;
(c) from about 1 mM to about 2 mM, to about 2.5 mM, to about 5 mM calcium chloride;
(d) from about 0 mM to about 10 mM, or about 20 mM, or about 30 mM, or about 40 mM, or about 50 mM sodium chloride;
(e) from about 20 ppm to about 50 ppm, or about 80 ppm, or about 100 ppm, or about 120 ppm, or about 200 ppm of a non-ionic surfactant;
(f) from about 0 mM to about 50 mM, or about 100 mM, or about 150 mM, or about 293 mM, or about 400 mM glycine; and
(g) from about 0.1 μg/ml to about 300 μg/ml, or about 1.0 μg/ml to about 200 μg/ml, or about 10 μg/ml to about 125 μg/ml of a rFVIII selected from a FL-rFVIII or a PEG-rFVIII selected from a PEG-FL-rFVIII, a PEG-BDD-rFVIII, and a PEG BDD-rFVIII-mutant;
   wherein the rFVIII formulation has a pH from about pH 6.0 to about pH 6.5, or about pH 7.0, or about pH 7.5.

Within still further aspects, the present disclosure provides rFVIII formulations, including FL-rFVIII formulations, as well as PEG-rFVIII formulations, including PEG-FL-rFVIII formulations, PEG-BDD-rFVIII formulations, and PEG BDD-rFVIII-mutant formulations, which rFVIII and PEG-rFVIII formulations comprise:
(a) from about 0 mM to about 20 mM or about 50 mM MOPS;
(b) from about 0 mM to about 29 mM, or about 34 mM, or about 58 mM, or about 100 mM, or about 300 mM of a sugar or sugar alcohol;
(c) from about 1 mM to about 2 mM, to about 2.5 mM, to about 5 mM calcium chloride;
(d) from about 0 mM to about 10 mM, or about 20 mM, or about 30 mM, or about 40 mM, or about 50 mM sodium chloride;
(e) from about 20 ppm to about 50 ppm, or about 80 ppm, or about 100 ppm, or about 120 ppm, or about 200 ppm of a non-ionic surfactant;
(f) from about 0 mM to about 50 mM, or about 100 mM, or about 150 mM, or about 293 mM, or about 400 mM glycine; and
(g) from about 0.1 μg/ml to about 300 μg/ml, or about 1.0 μg/ml to about 200 μg/ml, or about 10 μg/ml to about 125 μg/ml of a rFVIII selected from a FL-rFVIII or a PEG-rFVIII selected from a PEG-FL-rFVIII, a PEG-BDD-rFVIII, and a PEG BDD-rFVIII-mutant;
   wherein the rFVIII formulation has a pH from about pH 6.0 to about pH 6.5, or about pH 7.0, or about pH 7.5.

### BRIEF DESCRIPTION OF THE DRAWINGS

The skilled artisan will understand that the drawings, described below, are for illustration purposes only. The drawings are not intended to limit the scope of the disclosure provided herein or the scope of the claims in any way.

FIG. 1 is a graph showing the relative turbidity of BDD-rFVIII mutants with disulfide bond in buffer comprising increasing concentration of sodium chloride. Turbidity was measured by A₃₄₀ₙₘ. In addition to sodium chloride, the buffer comprised 20 mM histidine, 2.5 mM calcium chloride, 29 mM sucrose, 293 mM glycine and 80 ppm polysorbate 80.

FIG. 2 is a graph showing the relative turbidity of BDD-rFVIII mutants in buffer comprising increasing concentration of polysorbate 80. Turbidity was measured by A₃₄₀ₙₘ. In addition to polysorbate 80, the buffer comprised 20 mM histidine, 30 mM sodium chloride, 2.5 mM calcium chloride, 29 mM sucrose and 293 mM glycine.

FIG. 3 is a graph showing the relative turbidity of BDD-rFVIII mutants in buffer comprising increasing concentration of HSA. Turbidity was measured by A₃₄₀ₙₘ. The buffer comprised 20 mM histidine, 30 mM sodium chloride, 2.5 mM calcium chloride, 29 mM sucrose, 293 mM glycine and 80 ppm polysorbate 80.

FIG. 4 shows the relative turbidity of BDD-rFVIII mutants in a buffer comprising increasing concentration of sodium chloride in combination with polysorbate 80 and HSA. Turbidity was measured by A₃₄₀ₙₘ. In addition to sodium chloride, HSA and polysorbate 80, the buffer comprised 20 mM histidine, 2.5 mM calcium chloride, 29 mM sucrose and 293 mM glycine.

FIG. 5 shows clarity changes for BDD-rFVIII mutants with disulfide bond in solution before and after addition of excipients. From left to right: (1) combination of excipients (HSA, sodium chloride and polysorbate 80), (2) HSA, (3) sodium chloride, (4) polysorbate 80, and (5) before addition of HSA, polysorbate 80 and sodium chloride.

FIG. 6 is a graph showing liquid stability of full-length FVIII in histidine, MOPS and TEA buffers during 7 days storage at 40° C.

FIG. 7 is a graph showing rFVIII stability in MOPS and histidine buffer.

FIG. 8 is a diagram showing the structure of PEGylated BDD-rFVIII. The chains protruding from the A3 region represent the PEG molecule.

FIG. 9 is a graph showing the effect of sodium chloride on the potency recovery of PEGylated BDD-rFVIII during 6 days storage at 23° C.

FIG. 10 is a graph showing the effect of sodium chloride on the potency recovery of unPEGylated BDD-rFVIII during 6 days storage at 23° C.

FIG. 11 is a graph showing absorbance of PEGylated BDD-rFVIII in 25 mM, 55 mM, 75 mM, 125 mM and 200 mM NaCl/MOPS buffer.

FIG. 12 is a graph showing absorbance of unPEGylated BDD-rFVIII in 25 mM, 55 mM, 75 mM, 125 mM and 200 mM NaCl/MOPS buffer.

FIG. 13 is a graph showing normalized potency trends for PEGylated BDD-rFVIII in the platform formulation after 26 weeks.

FIG. 14 is a graph showing normalized potency trends for PEGylated BDD-rFVIII in the modified platform formulation after 26 weeks.

FIG. 15 is a graph showing normalized potency trends for PEGylated BDD-rFVIII in the sucrose formulation after 26 weeks.

FIG. 16 is a graph showing normalized potency trends for PEGylated BDD-rFVIII in the trehalose formulation after 26 weeks.

FIG. 17 is a graph showing normalized potency trends for PEGylated BDD-rFVIII in the platform formulation up to 30 months.

FIG. 18 is a graph showing normalized potency trends for PEGylated BDD-rFVIII in the modified platform formulation up to 13 weeks.

FIG. 19 is the amino acid sequence of BDD-rFVIII.

FIG. 20 is the amino acid sequence of FL-rFVIII.

### DESCRIPTION OF VARIOUS EMBODIMENTS

As described above, the present disclosure provides liquid and lyophilized rFVIII formulations, including full-length rFVIII (FL-rFVIII) formulations, B-domain deleted rFVIII (BDD-rFVIII) formulations, and BDD-rFVIII mutant (BDD-rVIII mutant) formulations. Also provided are liquid and lyophilized PEGylated rFVIII (PEG-rFVIII) formulations, including PEGylated full-length rFVIII (PEG-FL-rFVIII) formulations, PEGylated B-domain deleted rFVIII (PEG-BDD-rFVIII) formulations, and PEGylated BDD-rFVIII mutant (PEG-BDD-rFVIII mutant) formulations. As used herein, a BDD-rFVIII mutant comprises one or more cysteine substitution(s) within the amino acid sequence of BDD-rFVIII. Formulations described herein include one or more pharmaceutically acceptable excipients or stabilizers, and are comprised in buffered media at a suitable pH and osmolality suitable for in vivo administration.

For the purpose of interpreting this specification, the following definitions will apply. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). All references cited herein are incorporated by reference herein in their entirety.

Whenever appropriate, terms used in the singular also will include the plural and vice versa. The use of “a” herein means “one or more” unless stated otherwise or where the use of “one or more” is clearly inappropriate. The use of “or” means “and/or” unless stated otherwise. The use of “comprise,” “comprises,” “comprising,” “include,” “includes,” and “including” are interchangeable and not intended to be limiting. The terms “such as,” “for example,” and “e.g.” also are not intended to be limiting. For example, the term “including” shall mean “including, but not limited to.” Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” refers to +/−10% of the unit value provided. As used herein, the term “substantially” refers to the qualitative condition of exhibiting a total or approximate degree of a characteristic or property of interest. One of ordinary skill in the biological arts will understand that biological and chemical phenomena rarely, if ever, achieve or avoid an absolute result because of the many variables that affect testing, production, and storage of biological and chemical compositions and materials, and because of the inherent error in the instruments and equipment used in the testing, production, and storage of biological and chemical compositions and materials. The term substantially is therefore used herein to capture the potential lack of completeness inherent in many biological and chemical phenomena.

The presently disclosed rFVIII formulations, including FL-rFVIII formulations, BDD-rFVIII formulations, and BDD-rFVIII-mutant formulations, as well as PEG-rFVIII formulations, including PEG-FL-rFVIII formulations, PEG-BDD-rFVIII formulations, and PEG BDD-rFVIII-mutant formulations, which rFVIII and PEG-rFVIII formulations comprise: (a) from about 0 mM to about 20 mM or about 50 mM histidine; (b) from about 0 mM to about 29 mM, or about 34 mM, or about 58 mM, or about 100 mM, or about 300 mM of a sugar or sugar alcohol; (c) from about 1 mM to about 2 mM, to about 2.5 mM, to about 5 mM, to about 10 mM, to about 15 mM calcium chloride; (d) from about 100 mM to about 150 mM, or about 200 mM, or about 220 mM, or about 250 mM sodium chloride; (e) from about 20 ppm to about 50 ppm, or about 80 ppm, or about 100 ppm, or about 120 ppm, or about 200 ppm of a non-ionic surfactant; and (f) from about 0.1 μg/ml to about 300 μg/ml, or about 1.0 μg/ml to about 200 μg/ml, or about 10 μg/ml to about 125 μg/ml of a rFVIII selected from a FL-rFVIII, a BDD-rFVIII, and a BDD-rFVIII-mutant or a PEG-rFVIII selected from a PEG-FL-rFVIII, a PEG-BDD-rFVIII, and a PEG BDD-rFVIII-mutant; wherein the rFVIII formulation has a pH from about pH 6.0 to about pH 6.5, or about pH 7.0, or about pH 7.5.

Within certain aspects, these rFVIII formulations and PEG-rFVIII formulations comprise about 20 mM histidine, about 29 mM of a sugar or sugar alcohol, about 10 mM calcium chloride, about 220 mM sodium chloride, and about 100 ppm non-ionic surfactant.

Also provided are rFVIII formulations, including FL-rFVIII formulations, BDD-rFVIII formulations, and BDD-rFVIII-mutant formulations, as well as PEG-rFVIII formulations, including PEG-FL-rFVIII formulations, PEG-BDD-rFVIII formulations, and PEG BDD-rFVIII-mutant formulations, which rFVIII and PEG-rFVIII formulations comprise: (a) from about 0 mM to about 20 mM or about 50 mM MOPS; (b) from about 0 mM to about 29 mM, or about 34 mM, or about 58 mM, or about 100 mM, or about 300 mM of a sugar or sugar alcohol; (c) from about 1 mM to about 2 mM, to about 2.5 mM, to about 5 mM, to about 10 mM, to about 15 mM calcium chloride; (d) from about 100 mM to about 150 mM, or about 200 mM, or about 220 mM, or about 250 mM sodium chloride; (e) from about 20 ppm to about 50 ppm, or about 80 ppm, or about 100 ppm, or about 120 ppm, or about 200 ppm of a non-ionic surfactant; and (f) from about 0.1 μg/ml to about 300 μg/ml, or about 1.0 μg/ml to about 200 μg/ml, or about 10 μg/ml to about 125 μg/ml of a rFVIII selected from a FL-rFVIII, a BDD-rFVIII, and a BDD-rFVIII-mutant or a PEG-rFVIII selected from a PEG-FL-rFVIII, a PEG-BDD-rFVIII, and a PEG BDD-rFVIII-mutant; wherein the rFVIII formulation has a pH from about pH 6.0 to about pH 6.5, or about pH 7.0, or about pH 7.5.

Within certain aspects, these rFVIII formulations and PEG-rFVIII formulations comprise about 20 mM MOPS, about 29 mM of a sugar or sugar alcohol, about 10 mM calcium chloride, about 220 mM sodium chloride, and about 100 ppm non-ionic surfactant.

Further provided are rFVIII formulations, including FL-rFVIII formulations, as well as PEG-rFVIII formulations, including PEG-FL-rFVIII formulations, PEG-BDD-rFVIII formulations, and PEG BDD-rFVIII-mutant formulations, which rFVIII and PEG-rFVIII formulations comprise: (a) from about 0 mM to about 20 mM or about 50 mM histidine; (b) from about 0 mM to about 29 mM, or about 34 mM, or about 58 mM, or about 100 mM, or about 300 mM of a sugar or sugar alcohol; (c) from about 1 mM to about 2 mM, to about 2.5 mM, to about 5 mM calcium chloride; (d) from about 0 mM to about 10 mM, or about 20 mM, or about 30 mM, or about 40 mM, or about 50 mM sodium chloride; (e) from about 20 ppm to about 50 ppm, or about 80 ppm, or about 100 ppm, or about 120 ppm, or about 200 ppm of a non-ionic surfactant; (f) from about 0 mM to about 50 mM, or about 100 mM, or about 150 mM, or about 293 mM, or about 400 mM glycine; and (g) from about 0.1 μg/ml to about 300 μg/ml, or about 1.0 μg/ml to about 200 μg/ml, or about 10 μg/ml to about 125 μg/ml of a rFVIII selected from a FL-rFVIII or a PEG-rFVIII selected from a PEG-FL-rFVIII, a PEG-BDD-rFVIII, and a PEG BDD-rFVIII-mutant; wherein the rFVIII formulation has a pH from about pH 6.0 to about pH 6.5, or about pH 7.0, or about pH 7.5.

Within certain aspects, these rFVIII formulations and PEG-rFVIII formulations comprise about 20 mM histidine, about 29 mM of a sugar or sugar alcohol, about 2.5 mM calcium chloride, about 30 mM sodium chloride, about 80 ppm of a non-ionic surfactant, about 293 mM glycine, and about 50 μg/ml FL-rFVIII; wherein said FL-rFVIII formulation has a pH from about pH 6.5 to about pH 7.0.

Within other aspects, these rFVIII formulations and PEG-rFVIII formulations comprise about 20 mM histidine, about 37 mM of a sugar or sugar alcohol, about 2.5 mM calcium chloride, about 30 mM sodium chloride, about 80 ppm of a non-ionic surfactant, about 346 mM glycine, and about 50 μg/ml FL-rFVIII; wherein said FL-rFVIII formulation has a pH from about pH 6.5 to about pH 7.0.

Within further aspects, these rFVIII formulations and PEG-rFVIII formulations comprise about 20 mM histidine, from about 100 mM to about 300 mM of a sugar or sugar alcohol, about 2.5 mM calcium chloride, about 0 mM sodium chloride, about 80 ppm of a non-ionic surfactant, about 0 mM glycine, and about 14 μg/ml FL-rFVIII; wherein said FL-rFVIII formulation has a pH from about pH 6.5 to about pH 7.0.

Within still further aspects, these rFVIII formulations and PEG-rFVIII formulations comprise a sugar or sugar alcohol selected from sucrose and trehalose.

Within yet further aspects, these rFVIII formulations and PEG-rFVIII formulations comprise a non-ionic surfactant selected from polysorbate 20 and polysorbate 80.

Still further provided are rFVIII formulations, including FL-rFVIII formulations, as well as PEG-rFVIII formulations, including PEG-FL-rFVIII formulations, PEG-BDD-rFVIII formulations, and PEG BDD-rFVIII-mutant formulations, which rFVIII and PEG-rFVIII formulations comprise: (a) from about 0 mM to about 20 mM or about 50 mM MOPS; (b) from about 0 mM to about 29 mM, or about 34 mM, or about 58 mM, or about 100 mM, or about 300 mM of a sugar or sugar alcohol; (c) from about 1 mM, to about 2 mM, to about 2.5 mM, to about 5 mM calcium chloride; (d) from about 0 mM to about 10 mM, or about 20 mM, or about 30 mM, or about 40 mM, or about 50 mM sodium chloride; (e) from about 20 ppm to about 50 ppm, or about 80 ppm, or about 100 ppm, or about 120 ppm, or about 200 ppm of a non-ionic surfactant; (f) from about 0 mM to about 50 mM, or about 100 mM, or about 150 mM, or about 293 mM, or about 400 mM glycine; and (g) from about 0.1 μg/ml to about 300 μg/ml, or about 1.0 μg/ml to about 200 μg/ml, or about 10 μg/ml to about 125 μg/ml of a rFVIII selected from a FL-rFVIII or a PEG-rFVIII selected from a PEG-FL-rFVIII, a PEG-BDD-rFVIII, and a PEG BDD-rFVIII-mutant; wherein the rFVIII formulation has a pH from about pH 6.0 to about pH 6.5, or about pH 7.0, or about pH 7.5.

Within certain aspects, these rFVIII formulations and PEG-rFVIII formulations comprise about 20 mM MOPS, about 29 mM of a sugar or sugar alcohol, about 2.5 mM calcium chloride, about 30 mM sodium chloride, about 80 ppm of a non-ionic surfactant, about 293 mM glycine, and about 50 μg/ml FL-rFVIII; wherein said FL-rFVIII formulation has a pH from about pH 6.5 to about pH 7.0.

Within other aspects, these rFVIII formulations and PEG-rFVIII formulations comprise about 20 mM MOPS, about 37 mM of a sugar or sugar alcohol, about 2.5 mM calcium chloride, about 30 mM sodium chloride, about 80 ppm of a non-ionic surfactant, about 346 mM glycine, and about 50 μg/ml FL-rFVIII; wherein said FL-rFVIII formulation has a pH from about pH 6.5 to about pH 7.0.

Within further aspects, these rFVIII formulations and PEG-rFVIII formulations comprise about 20 mM MOPS, from about 100 mM to about 300 mM of a sugar or sugar alcohol, about 2.5 mM calcium chloride, about 0 mM sodium chloride, about 80 ppm of a non-ionic surfactant, about 0 mM glycine, and about 14 μg/ml FL-rFVIII; wherein said FL-rFVIII formulation has a pH from about pH 6.5 to about pH 7.0.

Within still further aspects, these rFVIII formulations and PEG-rFVIII formulations comprise a sugar or sugar alcohol selected from sucrose and trehalose.

Within yet further aspects, these rFVIII formulations and PEG-rFVIII formulations comprise a non-ionic surfactant selected from polysorbate 20 and polysorbate 80.

In these formulations, histidine and MOPS are buffer agents, which can be used to maintain the formulation pH from about pH 6.0 to about pH 7.5, or from about pH 6.5 to about pH 7.0, such as about pH 6.0, about pH 6.5, about pH 7.0, or about pH 7.5.

Sugars or sugar alcohols, such as mannitol, dextrose, glucose, trehalose, and/or sucrose, are used separately or in combination both as cryo-protectants and as stabilizers for the liquid formulations as well as during lyophilization.

As used herein, the term “osmolality” refers to a measure of solute concentration, defined as the number of osmoles of solute per kg of solvent. A desired level of osmolality can be achieved by the addition of one or more stabilizer such as a sugar or a sugar alcohol including mannitol, dextrose, glucose, trehalose, and/or sucrose. Additional stabilizers that are suitable for providing osmolality are described in references such as the handbook of Pharmaceutical Excipients (Fourth Edition, Royal Pharmaceutical Society of Great Britain, Science & Practice Publishers) or Remingtons: The Science and Practice of Pharmacy (Nineteenth Edition, Mack Publishing Company). Formulations described herein have an osmolality ranging from about 240 mOsm/kg to about 450 mOsm/kg, or about 750 mOsm/kg, or about 1000 mOsm/kg, or from about 270 mOsm/kg to about 425 mOsm/kg, or from about 300 mOsm/kg to about 410 mOsm/kg.

As used herein, the term “surfactant” includes “non-ionic surfactants” such as polysorbates including polysorbate 20 and polysorbate 80, polyoxamers including poloxamer 184 or 188, PLURONIC® polyols, and other ethylene/polypropylene block polymers. Non-ionic surfactants stabilize the rFVIII during processing and storage by reducing interfacial interaction and prevent protein from adsorption. The use of non-ionic surfactants permits the formulations to be exposed to shear and surface stresses without causing denaturation of the rFVIII. The formulations disclosed herein include formulations having one or more non-ionic surfactant(s), exemplified herein are formulations having a polysorbate, such as polysorbate 20 (Tween® 20) or polysorbate 80 (Tween® 80), which are present in formulations in the range of 20 ppm to 200 ppm.

Within certain aspects of the present disclosure, formulations comprising rFVIII and BDD-rFVIII, including formulations comprising PEGylated rFVIII and BDD-rFVIII, as well as formulations comprising mutant variants thereof can be lyophilized according to methodology known in the art. For example, U.S. Pat. Nos. 5,399,670 and 5,763,401 (incorporated by reference herein) describe methodology for producing lyophilized Factor VIII formulations of enhanced solubility, which methodology may be employed to lyophilize the formulations described herein. Table 1 provides exemplary process parameters for lyophilizing rFVIII and BDD-rFVIII formulations, including PEGylated rFVIII and BDD-rFVIII formulations. The lyophilization process has a freezing phase, a primary drying phase, and a secondary drying phase. In the freezing phase, there is an annealing step.

TABLE 1
Process Parameters for Lyophilizing rFVIII and BDD-rFVIII Formulations

| | | | | 1.25× | | |
|---|---|---|---|---|---|---|
| | | 1× Platform | | Platform | 1.5× Platform | |
| | Control | Limits | | Limits | Limits | |
| Process | Parameter | Lower | Upper | Upper | Lower | Upper |
| Parameter | Set point | Limit | Limit | Limit | Limit | Limit |
| Freezing rate | 0.3 | 0.2 | 0.4 | 0.4 | 0.1 | 0.5 |
| (° C./min) | | | | | | |
| Freezing | −45 | −48 | −41 | −42 | −50 | −40 |
| temperature (° C.) | | | | | | |
| Annealing warm- | 0.2 | 0.15 | 0.28 | 0.25 | 0.1 | 0.3 |
| up rate (° C./min) | | | | | | |
| Annealing | −20 | −23 | −16 | −17 | −25 | −15 |
| temperature (° C.) | | | | | | |
| Annealing cool- | 0.25 | 0.2 | 0.33 | 0.3 | 0.15 | 0.35 |
| down rate | | | | | | |
| (° C./min) | | | | | | |
| Final freezing | −45 | −48 | −41 | −42 | −50 | −40 |
| temperature (° C.) | | | | | | |
| Primary drying | 0.3 | 0.2 | 0.44 | 0.4 | 0.1 | 0.5 |
| warm-up rate | | | | | | |
| (° C./min) | | | | | | |
| Primary drying | −5 | −8 | −1 | −2 | −10 | 0 |
| temperature (° C.) | | | | | | |
| Secondary drying | 0.1 | 0.05 | 0.17 | 0.15 | 0.01 | 0.2 |
| warm-up rate | | | | | | |
| (° C./min) | | | | | | |
| Secondary drying | 25 | 22 | 29 | 28 | 20 | 30 |
| temperature (° C.) | | | | | | |
| Chamber Pressure | 100 | 50 | 150 | 162 | 25 | 175 |
| for Primary | | | | | | |
| Drying/Secondary | | | | | | |
| Drying (mTorr) | | | | | | |

As used herein, the term “biocompatible polymer” includes polyalkylene oxides such as without limitation polyethylene glycol (PEG), dextrans, colominic acids or other carbohydrate based polymers, polymers of amino acids, biotin derivatives, polyvinyl alcohol (PVA), polycarboxylates, polyvinylpyrrolidone, polyethylene-co-maleic acid anhydride, polystyrene-co-malic acid anhydride, polyoxazoline, polyacryloylmorpholine, heparin, albumin, celluloses, hydrolysates of chitosan, starches such as hydroxyethyl-starches and hydroxy propyl-starches, glycogen, agaroses and derivatives thereof, guar gum, pullulan, inulin, xanthan gum, carrageenan, pectin, alginic acid hydrolysates, other bio-polymers and any equivalents thereof. Preferred is polyethylene glycol, and still more preferred is methoxypolyethylene glycol (mPEG). Other useful polyalkylene glycol compounds are polypropylene glycols (PPG), polybutylene glycols (PBG), PEG-glycidyl ethers (Epox-PEG), PEG-oxycarbonylimidazole (CDI-PEG), branched polyethylene glycols, linear polyethylene glycols, forked polyethylene glycols and multi-armed or “super branched” polyethylene glycols (star-PEG).

As used herein, the terms “polyethylene glycol” or “PEG” are interchangeable and include any water-soluble poly(ethylene oxide). PEG includes the following structure “—(OCH₂CH₂)ₙ—” where (n) is 2 to 4000. As used herein, PEG also includes “—CH₂CH₂—O(CH₂CH₂O)ₙ—CH₂CH₂—” and “—(OCH₂CH₂)ₙO—,” depending upon whether or not the terminal oxygens have been displaced. The term “PEG” includes structures having various terminal or “end capping” groups, such as without limitation a hydroxyl or a C₁₋₂₀ alkoxy group. The term “PEG” also means a polymer that comprises a majority, that is to say, greater than 50%, of —OCH₂CH₂-repeating subunits. With respect to specific forms, the PEG can take any number of a variety of molecular weights, as well as structures or geometries such as branched, linear, forked, and multifunctional. As used herein, the term “PEGylation” refers to a process whereby a polyethylene glycol (PEG) is covalently attached to a molecule such as a protein. When a functional group such as a biocompatible polymer is described as activated, the functional group reacts readily with an electrophile or a nucleophile on another molecule.

The biocompatible polymer used in the conjugates disclosed herein may be any of the polymers discussed above. The biocompatible polymer is selected to provide the desired improvement in pharmacokinetics. For example, the identity, size and structure of the polymer is selected so as to improve the circulation half-life of the polypeptide having FVIII activity or decrease the antigenicity of the polypeptide without an unacceptable decrease in activity. The polymer can include PEG. For example, the polymer can be a polyethylene glycol terminally capped with an end-capping moiety such as hydroxyl, alkoxy, substituted alkoxy, alkenoxy, substituted alkenoxy, alkynoxy, substituted alkynoxy, aryloxy and substituted aryloxy. Alternatively, the polymer can include methoxypolyethylene glycol such as methoxypolyethylene glycol having a size range from 3 kD to 100 kD, and more preferably from 5 kD to 64 kD or from 5 kD to 43 kD.

The polymer can have a reactive moiety. For example, the polymer can have a sulfhydryl reactive moiety that can react with a free cysteine on a functional factor VIII polypeptide to form a covalent linkage. Such sulfhydryl reactive moieties include thiol, triflate, tresylate, aziridine, oxirane, S-pyridyl, or maleimide moieties. The polymer can be linear and include a “cap” at one terminus that is not strongly reactive towards sulfhydryls (such as methoxy) and a sulfhydryl reactive moiety at the other terminus. The conjugate can include PEG-maleimide having a size range from 5 kD to 64 kD.

As used herein, the full-length FVIII (rFVIII) refers to the blood clotting Factor VIII (FVIII), which is a glycoprotein synthesized and released into the bloodstream by the liver. In the circulating blood, it is bound to von Willebrand factor (vWF, also known as Factor VIII-related antigen) to form a stable complex. Upon activation by thrombin, it dissociates from the complex to interact with other clotting factors in the coagulation cascade, which eventually leads to the formation of a thrombus. Human full-length FVIII has the amino acid sequence of SEQ ID NO: 4, although allelic variants are possible.

As used herein, “functional factor VIII polypeptide” denotes a functional polypeptide or combination of polypeptides that are capable, in vivo or in vitro, of correcting human factor VIII deficiencies, characterized, for example, by hemophilia A. Factor VIII has multiple degradation or processed forms in the natural state. These are proteolytically derived from a precursor, one chain protein, as demonstrated herein. A functional factor VIII polypeptide includes such single chain protein and also provides for these various degradation products that have the biological activity of correcting human factor VIII deficiencies. Allelic variations likely exist. The functional factor VIII polypeptides include all such allelic variations, glycosylated versions, modifications and fragments resulting in derivatives of factor VIII so long as they comprise the functional segment of human factor VIII and the essential, characteristic human factor VIII functional activity remains unaffected in kind Those derivatives of factor VIII possessing the requisite functional activity can readily be identified by straightforward in vitro tests described herein. Furthermore, functional factor VIII polypeptide is capable of catalyzing the conversion of factor X to Xa in the presence of factor IXa, calcium, and phospholipid, as well as correcting the coagulation defect in plasma derived from hemophilia A affected individuals. From the disclosure of the sequence of the human factor VIII amino acid sequences and the functional regions herein, the fragments that can be derived via restriction enzyme cutting of the DNA or proteolytic or other degradation of human factor VIII protein will be apparent to those skilled in the art.

As used herein, B-domain deleted rFVIII (BDD-rFVIII) is characterized by having the amino acid sequence which comprises a deletion of all but 14 amino acids of the B-domain of FVIII. The first 4 amino acids of the B-domain (SFSQ, SEQ ID NO: 1) are linked to the 10 last residues of the B-domain (NPPVLKRHQR, SEQ ID NO: 2). Lind et al. Eur. J. Biochem. 232:19-27 (1995). The BDD-rFVIII used herein has the amino acid sequence of SEQ ID NO: 3.

As used herein the terms “rFVIII mutant” and “BDD rFVIII mutant” refers to genetically engineered protein variants of rFVIII and BDD rFVIII, respectively, which arise as a result of a laboratory induced mutation to a protein or polypeptide. It is envisioned that any functional factor VIII polypeptide may be mutated at a predetermined site and then covalently attached at that site to a biocompatible polymer according to the methods of the invention. Useful polypeptides include, without limitation, full-length factor VIII having the amino acid sequence as shown in SEQ ID NO: 4 and BDD rFVIII having the amino acid sequence as shown in SEQ ID NO: 3.

Site-directed mutation of a nucleotide sequence encoding polypeptide having FVIII activity may occur by any method known in the art. Methods include mutagenesis to introduce a cysteine codon at the site chosen for covalent attachment of the polymer. This may be accomplished using a commercially available site-directed mutagenesis kit such as the Stratagene cQuickChange™ II site-directed mutagenesis kit, the Clontech Transformer site-directed mutagenesis kit no. K1600-1, the Invitrogen GenTaylor site-directed mutagenesis system no. 12397014, the Promega Altered Sites II in vitro mutagenesis system kit no. Q6210, or the Takara Minis Bio LA PCR mutagenesis kit no. TAK RR016.

Conjugates described herein may be prepared by first replacing the codon for one or more amino acids on the surface of the functional FVIII polypeptide with a codon for cysteine, producing the cysteine mutant in a recombinant expression system, reacting the mutant with a cysteine-specific polymer reagent, and purifying the mutein. In this system, the addition of a polymer at the cysteine site can be accomplished through a maleimide active functionality on the polymer.

The amount of sulfhydryl reactive polymer used should be at least equimolar to the molar amount of cysteines to be derivatized and preferably is present in excess. A 5-fold or a 10-fold molar excess of sulfhydryl reactive polymer can be used. Other conditions useful for covalent attachment are within the skill of those in the art.

The rFVIII and BDD-rFVIII mutants disclosed herein are named in a manner conventional in the art. The convention for naming mutants is based on the amino acid sequence for the mature, full length Factor VIII as provided in SEQ ID NO: 4. As a secreted protein, FVIII comprises a signal sequence that is proteolytically cleaved during the translation process. Following removal of the 19 amino acid signal sequence, the first amino acid of the secreted FVIII product is an alanine.

As is conventional and as used herein, when referring to mutated amino acids in BDD rFVIII, the mutated amino acid is designated by its position in the sequence of full-length FVIII. For example, a BDD rFVIII mutant can include a K1808C amino acid substitution wherein the lysine (K) at the position analogous to 1808 in the full-length sequence is substituted to cysteine (C).

The predefined site for covalent binding of the polymer, e.g., PEG, can be selected from sites exposed on the surface of the rFVIII or BDD rFVIII polypeptide that are not involved in FVIII activity or involved in other mechanisms that stabilize FVIII in vivo, such as binding to vWF. Such sites are also best selected from those sites known to be involved in mechanisms by which FVIII is deactivated or cleared from circulation. Sites for substituting an amino acid with a cysteine include an amino acid residue in or near a binding site for (a) low density lipoprotein receptor related protein, (b) a heparin sulphate proteoglycan, (c) low density lipoprotein receptor and/or (d) factor VIII inhibitory antibodies. By “in or near a binding site” means a residue that is sufficiently close to a binding site such that covalent attachment of a biocompatible polymer to the site would result in steric hindrance of the binding site. Such a site is expected to be within 20 \acute{Å} of a binding site, for example.

The biocompatible polymer can be covalently attached to the functional factor VIII polypeptide at an amino acid residue in or near (a) a factor VIII clearance receptor as defined supra, (b) a binding site for a protease capable of degradation of factor VIII and/or (c) a binding site for factor VIII inhibitory antibodies. The protease may be activated protein C (APC). The biocompatible polymer can be covalently attached at the predefined site on the functional factor VIII polypeptide such that binding of low-density lipoprotein receptor related protein to the polypeptide is less than to the polypeptide when it is not conjugated, and preferably more than twofold less. The biocompatible polymer can be covalently attached at the predefined site on the functional factor VIII polypeptide such that binding of heparin sulphate proteoglycans to the polypeptide is less than to the polypeptide when it is not conjugated, and preferably is more than twofold less. The biocompatible polymer can be covalently attached at the predefined site on the functional factor VIII polypeptide such that binding of factor VIII inhibitory antibodies to the polypeptide is less than to the polypeptide when it is not conjugated. The biocompatible polymer can be covalently attached at the predefined site on the functional factor VIII polypeptide such that binding of low density lipoprotein receptor to the polypeptide is less than to the polypeptide when it is not conjugated. The biocompatible polymer can be covalently attached at the predefined site on the functional factor VIII polypeptide such that a plasma protease degrades the polypeptide less than when the polypeptide is not conjugated.

The biocompatible polymer can be covalently attached to the rFVIII or BDD rFVIII polypeptide, or mutant variant thereof, at one or more of the factor VIII amino acid positions 81, 129, 377, 378, 468, 487, 491, 504, 556, 570, 711, 1648, 1795, 1796, 1803, 1804, 1808, 1810, 1864, 1903, 1911, 2091, 2118 and 2284. Alternatively, the biocompatible polymer can be covalently attached to the polypeptide at one or more of factor VIII amino acid positions 377, 378, 468, 491, 504, 556, 1795, 1796, 1803, 1804, 1808, 1810, 1864, 1903, 1911 and 2284; or to the polypeptide at one or more of factor VIII amino acid positions 377, 378, 468, 491, 504, 556 and 711; or to one or more of the factor VIII amino acid positions 81, 129, 377, 378, 468, 487, 491, 504, 556, 570, 711, 1648, 1795, 1796, 1803, 1804, 1808, 1810, 1864, 1903, 1911, 2091, 2118 and 2284; or to one or more of the factor VIII amino acid positions 81, 129, 377, 378, 468, 487, 491, 504, 556, 570, 711, 1648, 1795, 1796, 1803, 1804, 1808, 1810, 1864, 1903, 1911, 2091, 2118 and 2284; or to one or more of positions 377, 378, 468, 491, 504, 556, and 711.

The biocompatible polymer can be covalently attached to B-domain deleted rFVIII at amino acid position 129, 491, 1804, and/or 1808. One or more sites, preferably one or two, on the functional factor VIII polypeptide may be the predefined sites for polymer attachment. In particular embodiments, the polypeptide is mono-PEGylated or diPEGylated.

The rFVIII and BDD rFVIII conjugates described herein can be made by mutating a nucleotide sequence that encodes for a functional factor VIII polypeptide to substitute a coding sequence for a cysteine residue at a pre-defined site; expressing the mutated nucleotide sequence to produce a cysteine enhanced mutant; purifying the mutant; reacting the mutant with the biocompatible polymer (e.g., PEG) that has been activated to react with polypeptides at substantially only reduced cysteine residues such that the conjugate is formed; and purifying the conjugate.

Site directed PEGylation of a factor VIII mutant can also be achieved by: (a) expressing a site-directed factor VIII mutant wherein the mutant has a cysteine replacement for an amino acid residue on the exposed surface of the factor VIII mutant and that cysteine is capped; (b) contacting the cysteine mutant with a reductant under conditions to mildly reduce the cysteine mutant and to release the cap; (c) removing the cap and the reductant from the cysteine mutant; and (d) after the removal of the reductant, treating the cysteine mutant with PEG comprising a sulfhydryl coupling moiety under conditions such that PEGylated factor VIII mutein is produced. The sulfhydryl coupling moiety of the PEG is selected from the group consisting of thiol, triflate, tresylate, aziridine, oxirane, S-pyridyl and maleimide moieties, preferably maleimide.

The present disclosure also provides methods for the treatment of hemophilia A in a patient, comprising the administration to the patient of a therapeutically effective amount of one or more formulations described herein. These formulations may be administrated to a patient via intravenous injection, subcutaneous injection, or through continuous infusion.

Within certain aspects are provided methods for the treatment of hemophilia A in a patient, comprising the administration to the patient of a therapeutically effective amount of a rFVIII formulations, including FL-rFVIII formulations, BDD-rFVIII formulations, and BDD-rFVIII-mutant formulations, as well as PEG-rFVIII formulations, including PEG-FL-rFVIII formulations, PEG-BDD-rFVIII formulations, and PEG BDD-rFVIII-mutant formulations, which rFVIII and PEG-rFVIII formulations comprise: (a) from about 0 mM to about 20 mM or about 50 mM histidine; (b) from about 0 mM to about 29 mM, or about 34 mM, or about 58 mM, or about 100 mM, or about 300 mM of a sugar or sugar alcohol; (c) from about 1 mM to about 2 mM, to about 2.5 mM, to about 5 mM, to about 10 mM, to about 15 mM calcium chloride; (d) from about 100 mM to about 150 mM, or about 200 mM, or about 220 mM, or about 250 mM sodium chloride; (e) from about 20 ppm to about 50 ppm, or about 80 ppm, or about 100 ppm, or about 120 ppm, or about 200 ppm of a non-ionic surfactant; and (f) from about 0.1 μg/ml to about 300 μg/ml, or about 1.0 μg/ml to about 200 μg/ml, or about 10 μg/ml to about 125 μg/ml of a rFVIII selected from a FL-rFVIII, a BDD-rFVIII, and a BDD-rFVIII-mutant or a PEG-rFVIII selected from a PEG-FL-rFVIII, a PEG-BDD-rFVIII, and a PEG BDD-rFVIII-mutant; wherein the rFVIII formulation has a pH from about pH 6.0 to about pH 6.5, or about pH 7.0, or about pH 7.5.

Also provided are methods for the treatment of hemophilia A in a patient, comprising the administration to the patient of a therapeutically effective amount of a rFVIII formulations, including FL-rFVIII formulations, as well as PEG-rFVIII formulations, including PEG-FL-rFVIII formulations, PEG-BDD-rFVIII formulations, and PEG BDD-rFVIII-mutant formulations, which rFVIII and PEG-rFVIII formulations comprise: (a) from about 0 mM to about 20 mM or about 50 mM histidine; (b) from about 0 mM to about 29 mM, or about 34 mM, or about 58 mM, or about 100 mM, or about 300 mM of a sugar or sugar alcohol; (c) from about 1 mM to about 2 mM, to about 2.5 mM, to about 5 mM calcium chloride; (d) from about 0 mM to about 10 mM, or about 20 mM, or about 30 mM, or about 40 mM, or about 50 mM sodium chloride; (e) from about 20 ppm to about 50 ppm, or about 80 ppm, or about 100 ppm, or about 120 ppm, or about 200 ppm of a non-ionic surfactant; (f) from about 0 mM to about 50 mM, or about 100 mM, or about 150 mM, or about 293 mM, or about 400 mM glycine; and (g) from about 0.1 μg/ml to about 300 μg/ml, or about 1.0 μg/ml to about 200 μg/ml, or about 10 μg/ml to about 125 μg/ml of a rFVIII selected from a FL-rFVIII or a PEG-rFVIII selected from a PEG-FL-rFVIII, a PEG-BDD-rFVIII, and a PEG BDD-rFVIII-mutant; wherein the rFVIII formulation has a pH from about pH 6.0 to about pH 6.5, or about pH 7.0, or about pH 7.5.

As used herein, the term “therapeutically effective amount” of a rFVIII formulation or a PEGylated rFVIII formulation refers to an amount of the formulation that provides therapeutic effect in an administration regimen. Because of their low viscosity, the presently disclosed rFVIII and PEG-rFVIII formulations can be conveniently processed via, for example, ultrafiltration and sterile filtration and can be administered to a patient via injection, including intravenous injection, subcutaneous injection, and continuous infusion.

Moreover, because they have an osmolality ranging from about 240 mOsm/kg to about 450 mOsm/kg, or about 750 mOsm/kg, or about 1000 mOsm/kg, or from about 270 mOsm/kg to about 425 mOsm/kg, or from about 300 mOsm/kg to about 410 mOsm/kg, or from about 300 mmol/kg to about 410 mmol/kg, the presently disclosed rFVIII and PEG-rFVIII formulations reduce tissue damage or other adverse physiologic effects, which increases favorable patient tolerance and patient compliance.

Aspects of the present disclosure may be further understood in light of the following examples, which should not be construed as limiting the scope of the present teachings in any way.

### EXAMPLES

### Example 1

### Effect of Sodium Chloride, Polysorbate 80, and Human Serum Albumin on BDD-rFVIII Protein Solubility and Stability

Effect of Sodium Chloride

A study was performed to determine whether the precipitation observed when BDD-rFVIII mutants were placed in histidine buffer could be reversed. The buffer solution comprised 20 mM histidine, 30 mM sodium chloride, 2.5 mM calcium chloride, 29 mM sucrose, 293 mM glycine and 80 ppm polysorbate 80. The aim of the study was to develop a formulation that stabilized BDD-rFVIII mutants. Commonly used solubilizers and stabilizers, such as sodium chloride, Polysorbate 80, and human serum albumin (HSA) were tested to either increase the solubility of the mutants or to improve the stability by reducing protein aggregation. The study established that as the sodium chloride concentration increased, the turbidity of the solution comprising the mutants decreased, suggesting that sodium chloride reversed the precipitation process. When the sodium chloride concentration was 176 mM or higher, the cloudy solution turned to a clear solution and the turbidity dropped from 0.169 to 0.029, which is more than 80% based on A_{340 nm} measurements (FIG. 1). These results demonstrated that sodium chloride was an effective solubilizer for the BDD-rFVIII mutants and can reverse their precipitation.

In summary, higher sodium chloride concentrations improved the solubility of the BDD-rFVIII mutants.

TABLE 2
Formulation Composition for
full-length rFVIII and BDD-rFVIII mutants

| | BDD- | Full-length |
|---|---|---|
| Composition | rFVIIImutants | rFVIII |
| Sodium chloride (mM) | 220 | 30 |
| Sucrose (mM) | 29 | 29 |
| Histidine (mM) | 20 | 20 |
| Glycine (mM) | 293 | 293 |
| Calcium chloride (mM) | 2.5 | 2.5 |
| Polysorbate 80 (ppm) | 80 | 80 |

### Example 2

### Formulation Development for rFVIII PEGylation Through Random Lysine Coupling

PEG polymer was conjugated to the full-length rFVIII and BDD-rFVIII using random lysine coupling. In this type of coupling, the reactive groups are primarily the N-terminal amine or the ε-amino group of lysine in a protein. Therefore, any other primary or secondary amine groups in the formulation could interfere with the reaction. Since full-length and BDD-rFVIII formulations comprise amino acids, such as glycine and histidine, new formulations were developed for PEGylation of these molecules. While glycine was used as a bulking agent in the full-length rFVIII formulation and could be eliminated during PEGylation, histidine served as a buffer component and needed to be replaced with another buffer.

A suitable buffer system meets the following criteria: (1) it provides buffer capacity at pH 6-7; (2) it does not form insoluble complex or chelate with calcium chloride, an important rFVIII stabilizer; and (3) it does not comprise primary or secondary amine groups.

Several commonly used buffers were considered for random PEGylation of rFVIII. As shown in Table 3, only two buffer systems, tri-ethanolamine (TEA) and 3-[N-Morpholino]propanesulfonic acid (MOPS) were selected for further investigation.

TABLE 3
Buffers Considered for Random PEGylation of rFVIII

| | | | | pH change |
|---|---|---|---|---|
| Buffer at pH | Ca²⁺ | Ca²⁺ | Amine | during |
| 7 | ppt. | chelating | group | freezing |
| Citrate | | X | | |
| Phosphate | X | | | X |
| Histidine | | | X | |
| TRIS | | | X | |
| Carbonate | X | | | |
| Triethanolamine | | | | |
| (TEA) | | | | |
| MOPS or MOPSO | | | | |
| HEPES | | | X | |

For this study, full-length rFVIII was dialyzed against the formulations listed in Table 4. The dialyzed rFVIII in the three formulations was placed at 40° C. to establish stability at accelerated conditions and the results are shown in FIGS. 6 and 7.

TABLE 4
Buffers Evaluated for Random PEGylation of rFVIII

| | | | | | | Sodium | Buffer |
|---|---|---|---|---|---|---|---|
| | NaCl | CaCl₂ | Tween 80 | Glycine | Sucrose | Azide | Agent |
| | (mM) | (mM) | (ppm) | (mM) | (mM) | (%) | (20 mM) |
| 1 | 30 | 2.5 | 80 | — | 29 | 0.05 | TEA |
| 2 | 30 | 2.5 | 80 | — | 29 | 0.05 | MOPS |
| 3 | 30 | 2.5 | 80 | 293 | 29 | 0.05 | Histidine |

### Example 3

### PEGylation for BDD-rFVIII

As discussed above, BDD-rFVIII encounters formulation challenges due to its propensity for aggregation. Therefore, one of the objectives with designing a formulation for PEGylated rFVIII was to ensure its stability in solution. The working formulation for the PEGylated BDD-rFVIII comprised 200 mM sodium chloride, 20 mM MOPS, 10 mM CaCl₂, 100 ppm polysorbate 80 and 29 mM sucrose. 200 mM sodium chloride will impose difficulties during freeze-drying. Accordingly, the solubility and potency of the PEGylated BDD-rFVIII was evaluated as a function of sodium chloride concentration in the range of 50 and 250 mM. Since the PEGylated rFVIII molecule is more hydrophilic than BDD-rFVIII, the optimum sodium chloride concentration for maintaining rFVIII in solution should be below 200 mM.

The buffer composition used for the study is shown in Table 5 and the data are summarized in FIGS. 9 and 10. The PEGylated BDD-rFVIII retained more than 87% potency in the formulation comprising 50-150 mM sodium chloride during 6 days storage at 23° C. UnPEGylated BDD-rFVIII retained 70% potency in the same formulation during 6 days storage at 23° C. Both molecules remained soluble during the study with no visual detection of precipitates or opalescence. These and earlier data suggest that 100 mM sodium chloride can be used for further formulation development.

TABLE 5
Composition of the Formulation Used
for Evaluating the Effect of Sodium Chloride

| | | | Polysorbate | |
|---|---|---|---|---|
| MOPS | NaCl | CaCl2 | 80 | Sucrose |
| (mM) | (mM) | (mM) | (Ppm) | (mM) |
| 20 | 250 | 10 | 100 | 29 |
| 20 | 200 | 10 | 100 | 29 |
| 20 | 150 | 10 | 100 | 29 |
| 20 | 100 | 10 | 100 | 29 |
| 20 | 50 | 10 | 100 | 29 |
| 20 | 25 | 10 | 100 | 29 |
| 20 | 0 | 10 | 100 | 29 |

The effect of sodium chloride on the solubility and aggregation of PEGylated and unPEGylated BDD-rFVIII was investigated.

FIG. 11 shows the UV absorbance of PEGylated BDD-rFVIII in MOPS buffer comprising 25 mM, 55 mM, 75 mM, 125 mM and 200 mM sodium chloride. UV absorbance results showed no scattering of the PEGylated BDD-rFVIII at all sodium chloride concentration tested, suggesting lack of aggregation. In contrast, the unPEGylated-rFVIII showed considerable scattering at 25 mM, 55 mM and 75 mM sodium chloride (FIG. 12) most likely due to formation of soluble aggregates. When sodium chloride concentration was increased to 125 mM and 200 mM, no scattering was observed. It was concluded, therefore, that higher salt concentrations prevented aggregate formation.

### Example 4

### Development of Freeze-Drying Formulation for PEGylated BDD-rFVIII

Four candidate formulations were screened with the PEGylated BDD-rFVIII. The aim was to evaluate the stability of the lyophilized drug product in these formulations and to select a formulation for the leading stability study. The formulations that were screened were (1) platform formulation used for full-length rFVIII, (2) modified platform formulation, comprising increased solids content compared to the platform formulation, (3) sucrose formulation, and (4) trehalose formulation. The last two formulations provided an amorphous matrix for the lyophilized drug product.

Stability was evaluated at three storage temperatures (5° C., 25° C. and 40° C.). Table 6 shows the formulation composition for PEGylated BDD-rFVIII used for stability evaluation.

The concentrations of sucrose and glycine were increased from 29 mM and 293 mM in the platform formulation to 38 mM and 346 mM in the modified platform formulation. The additional solids were added to enhance the mechanical strength of the freeze-dried cake and improve the appearance of the final drug product.

TABLE 6
Formulation Composition for PEGylated
BDD-rFVIII Used in Stability Evaluation¹

| | | Modified | | |
|---|---|---|---|---|
| | Platform | Platform | Sucrose | Trehalose |
| Component | Formulation | Formulation | Formulation | Formulation |
| Calcium | 2.5 mM | 2.5 mM | 2.5 mM | 2.5 mM |
| Chloride | | | | |
| Sodium | 30 mM | 30 mM | X | X |
| Chloride | | | | |
| Histidine | 20 mM | 20 mM | 20 mM | 20 mM |
| Glycine | 293 mM | 346 mM | X | X |
| Polysorbate | 80 ppm | 80 ppm | 80 ppm | 80 ppm |
| 80 | | | | |
| Sucrose | 29 mM | 38 mM | 0.234 mM | X |
| Trehalose | X | X | X | 0.211 mM |

¹pH = 6.8 for all formulations

The sucrose and trehalose formulations were designed to provide an alternate matrix compared to the other two formulations. The platform and modified platform formulations formed a crystalline matrix upon freeze-drying due to the presence of sodium chloride and glycine as structural stability and bulking agents. The concentrations of sucrose and trehalose were increased to 234 mM and 211 mM, the respectively, in lieu of including sodium chloride and glycine. This resulted in an amorphous matrix for the freeze-dried drug product.

The stability program for each of the four candidate formulations was set up for a 26 week time period. Stability was evaluated by potency, moisture content and total protein. The potency recovery data for the four formulations are summarized in FIGS. 13-16.

The data showed potency recovery, moisture content by Karl Fischer, and percent aggregates and product related impurities by SEC-HPLC (tested at 26 weeks) for the four formulations in the expected range.

Stability for PEGylated BDD-rFVIII was further evaluated with the platform and modified platform formulations (see Table 6 for modified formulation compositions). Two drug product lots were prepared at lab-scale and were placed on stability at 5° C. and 25° C. and 40° C. Potency by the chromogenic assay, percent aggregates and total protein by SEC-HPLC, and moisture by Karl Fischer were employed for drug product stability evaluation.

These data demonstrated comparable drug product stability in the two formulations. The study with the platform formulation was continued up to 30 months, whereas the study with the modified formulation was terminated at 3 months (FIGS. 17 and 18).

### REFERENCES

1. Roberts, M. J.; Bentley, M. D. and Harris, J. M. Chemistry for peptide and protein PEGylation. Advanced Drug Delivery Reviews 2002, 54, 459-476
2. Björkman, S and Berntorp, E. Pharmacokinetics of coagulation factors. Clinical Pharmacokinetics 2001 40, 815-832
3. Mei, B., Pan, C., Jiang, H., Tjiandra, H., Strauss, J., Chen, Y., Liu, T., Zhang, X., Severs, J., Newgren, J., Chen, J., Gu, J. M., Subramanyam, B., Fournel, M. A., Pierce, J. F., Murphy, J. E. Rational design of a fully active, long-acting PEGylated Factor VIII for hemophilia A treatment. Blood 2010, 116, 270-279.

What is claimed is:

1. A full-length rFVIII (FL-rFVIII) formulation, comprising:
a. from about 0 mM to about 20 mM or about 50 mM histidine;
b. from about 0 mM to about 29 mM, or about 34 mM, or about 58 mM, or about 100 mM, or about 300 mM of a sugar or sugar alcohol;
c. from about 1 mM to about 2 mM, or about 2.5 mM, or about 5 mM, or about 10 mM, or about 15 mM calcium chloride;
d. from about 100 mM to about 150 mM, or about 200 mM, or about 220 mM, or about 250 mM sodium chloride;
e. from about 20 ppm to about 50 ppm, or about 80 ppm, or about 100 ppm, or about 120 ppm, or about 200 ppm of a non-ionic surfactant; and
f. from about 0.1 μg/ml to about 300 μg/ml, or from about 1.0 μg/ml to about 200 μg/ml, or from about 10 μg/ml to about 125 μg/ml of a FL-rFVIII;
wherein said FL-rFVIII formulation has a pH from about pH 6.0 to about pH 6.5, or about pH 7.0, or about pH 7.5.

2. The FL-rFVIII formulation of claim 1, comprising: about 20 mM histidine, about 29 mM of a sugar or sugar alcohol, about 10 mM calcium chloride, about 220 mM sodium chloride, and about 100 ppm non-ionic surfactant.

3. A B-domain deleted rFVIII (BDD-rFVIII) formulation, comprising:
a. from about 0 mM to about 20 mM or about 50 mM histidine;
b. from about 0 mM to about 29 mM, or about 34 mM, or about 58 mM, or about 100 mM, or about 300 mM of a sugar or sugar alcohol;
c. from about 1 mM to about 2 mM, or about 2.5 mM, or about 5 mM, or about 10 mM, or about 15 mM calcium chloride;
d. from about 100 mM to about 150 mM, or about 200 mM, or about 220 mM, or about 250 mM sodium chloride;
e. from about 20 ppm to about 50 ppm, or about 80 ppm, or about 100 ppm, or about 120 ppm, or about 200 ppm of a non-ionic surfactant; and
f. from about 0.1 μg/ml to about 300 μg/ml, or from about 1.0 μg/ml to about 200 μg/ml, or from about 10 μg/ml to about 125 μg/ml of a BDD-rFVIII;
wherein said BDD-rFVIII formulation has a pH from about pH 6.0 to about pH 6.5, or about pH 7.0, or about pH 7.5.

4. The BDD-rFVIII formulation of claim 3, comprising: about 20 mM histidine, about 29 mM of a sugar or sugar alcohol, about 10 mM calcium chloride, about 220 mM sodium chloride, and about 100 ppm non-ionic surfactant.

5. A B-domain deleted rFVIII mutant (BDD-rFVIII-mutant) formulation, comprising:
a. from about 0 mM to about 20 mM or about 50 mM histidine;
b. from about 0 mM to about 29 mM, or about 34 mM, or about 58 mM, or about 100 mM, or about 300 mM of a sugar or sugar alcohol;
c. from about 1 mM to about 2 mM, or about 2.5 mM, or about 5 mM, or about 10 mM, or about 15 mM calcium chloride;
d. from about 100 mM to about 150 mM, or about 200 mM, or about 220 mM, or about 250 mM sodium chloride;
e. from about 20 ppm to about 50 ppm, or about 80 ppm, or about 100 ppm, or about 120 ppm, or about 200 ppm of a non-ionic surfactant; and
f. from about 0.1 μg/ml to about 300 μg/ml, or from about 1.0 μg/ml to about 200 μg/ml, or from about 10 μg/ml to about 125 μg/ml of a BDD-rFVIII-mutant;
wherein said BDD-rFVIII-mutant formulation has a pH from about pH 6.0 to about pH 6.5, or about pH 7.0, or about pH 7.5.

6. The BDD-rFVIII-mutant formulation of claim 5, comprising: about 20 mM histidine, about 29 mM of a sugar or sugar alcohol, about 10 mM calcium chloride, about 220 mM sodium chloride, and about 100 ppm non-ionic surfactant.

7. A PEGylated full-length rFVIII (PEG FL-rFVIII) formulation, comprising:
a. from about 0 mM to about 20 mM or about 50 mM histidine;
b. from about 0 mM to about 29 mM, or about 34 mM, or about 58 mM, or about 100 mM, or about 300 mM of a sugar or sugar alcohol;
c. from about 1 mM to about 2 mM, or about 2.5 mM, or about 5 mM, or about 10 mM, or about 15 mM calcium chloride;
d. from about 100 mM to about 150 mM, or about 200 mM, or about 220 mM, or about 250 mM sodium chloride;
e. from about 20 ppm to about 50 ppm, or about 80 ppm, or about 100 ppm, or about 120 ppm, or about 200 ppm of a non-ionic surfactant; and
f. from about 0.1 μg/ml to about 300 μg/ml, or from about 1.0 μg/ml to about 200 μg/ml, or from about 10 μg/ml to about 125 μg/ml of a PEG FL-rFVIII;
wherein said PEG FL-rFVIII formulation has a pH from about pH 6.0 to about pH 6.5, or about pH 7.0, or about pH 7.5.

8. The PEG FL-rFVIII formulation of claim 7, comprising: about 20 mM histidine, about 29 mM of a sugar or sugar alcohol, about 10 mM calcium chloride, about 220 mM sodium chloride, and about 100 ppm non-ionic surfactant.

9. A PEGylated B-domain deleted rFVIII (PEG-BDD-rFVIII) formulation, comprising:
a. from about 0 mM to about 20 mM or about 50 mM histidine;
b. from about 0 mM to about 29 mM, or about 34 mM, or about 58 mM, or about 100 mM, or about 300 mM of a sugar or sugar alcohol;
c. from about 1 mM to about 2 mM, or about 2.5 mM, or about 5 mM, or about 10 mM, or about 15 mM calcium chloride;
d. from about 100 mM to about 150 mM, or about 200 mM, or about 220 mM, or about 250 mM sodium chloride;
e. from about 20 ppm to about 50 ppm, or about 80 ppm, or about 100 ppm, or about 120 ppm, or about 200 ppm of a non-ionic surfactant; and
f. from about 0.1 μg/ml to about 300 μg/ml, or from about 1.0 μg/ml to about 200 μg/ml, or from about 10 μg/ml to about 125 μg/ml of a PEG-BDD-rFVIII;
wherein said PEG-BDD-rFVIII formulation has a pH from about pH 6.0 to about pH 6.5, or about pH 7.0, or about pH 7.5.

10. The PEG-BDD-rFVIII formulation of claim 9, comprising: about 20 mM histidine, about 29 mM of a sugar or sugar alcohol, about 10 mM calcium chloride, about 220 mM sodium chloride, and about 100 ppm non-ionic surfactant.

11. A PEG BDD-rFVIII-mutant formulation, comprising:
a. from about 0 mM to about 20 mM or about 50 mM histidine;
b. from about 0 mM to about 29 mM, or about 34 mM, or about 58 mM, or about 100 mM, or about 300 mM of a sugar or sugar alcohol;
c. from about 1 mM to about 2 mM, or about 2.5 mM, or about 5 mM, or about 10 mM, or about 15 mM calcium chloride;
d. from about 100 mM to about 150 mM, or about 200 mM, or about 220 mM, or about 250 mM sodium chloride;
e. from about 20 ppm to about 50 ppm, or about 80 ppm, or about 100 ppm, or about 120 ppm, or about 200 ppm of a non-ionic surfactant; and
f. from about 0.1 μg/ml to about 300 μg/ml, or from about 1.0 μg/ml to about 200 μg/ml, or from about 10 μg/ml to about 125 μg/ml of a PEG BDD-rFVIII-mutant;
wherein said PEG BDD-rFVIII-mutant formulation has a pH from about pH 6.0 to about pH 6.5, or about pH 7.0, or about pH 7.5.

12. The PEG BDD-rFVIII-mutant formulation of claim 11, comprising: about 20 mM histidine, about 29 mM of a sugar or sugar alcohol, about 10 mM calcium chloride, about 220 mM sodium chloride, and about 100 ppm non-ionic surfactant.

13. A full-length rFVIII (FL-rFVIII) formulation, comprising:
a. from about 0 mM to about 20 mM or about 50 mM MOPS;
b. from about 0 mM to about 29 mM, or about 34 mM, or about 58 mM, or about 100 mM, or about 300 mM of a sugar or sugar alcohol;
c. from about 1 mM to about 2 mM, or about 2.5 mM, or about 5 mM, or about 10 mM, or about 15 mM calcium chloride;
d. from about 100 mM to about 150 mM, or about 200 mM, or about 220 mM, or about 250 mM sodium chloride;
e. from about 20 ppm to about 50 ppm, or about 80 ppm, or about 100 ppm, or about 120 ppm, or about 200 ppm of a non-ionic surfactant; and
f. from about 0.1 μg/ml to about 300 μg/ml, or from about 1.0 μg/ml to about 200 μg/ml, or from about 10 μg/ml to about 125 μg/ml of a FL-rFVIII;
wherein said FL-rFVIII formulation has a pH from about pH 6.0 to about pH 6.5, or about pH 7.0, or about pH 7.5.

14. The FL-rFVIII formulation of claim 13, comprising: about 20 mM MOPS, about 29 mM of a sugar or sugar alcohol, about 10 mM calcium chloride, about 220 mM sodium chloride, and about 100 ppm non-ionic surfactant.

15. A B-domain deleted rFVIII (BDD-rFVIII) formulation, comprising:
a. from about 0 mM to about 20 mM or about 50 mM MOPS;
b. from about 0 mM to about 29 mM, or about 34 mM, or about 58 mM, or about 100 mM, or about 300 mM of a sugar or sugar alcohol;
c. from about 1 mM to about 2 mM, or about 2.5 mM, or about 5 mM, or about 10 mM, or about 15 mM calcium chloride;
d. from about 100 mM to about 150 mM, or about 200 mM, or about 220 mM, or about 250 mM sodium chloride;
e. from about 20 ppm to about 50 ppm, or about 80 ppm, or about 100 ppm, or about 120 ppm, or about 200 ppm of a non-ionic surfactant; and
f. from about 0.1 μg/ml to about 300 μg/ml, or from about 1.0 μg/ml to about 200 μg/ml, or from about 10 μg/ml to about 125 μg/ml of a BDD-rFVIII;
wherein said BDD-rFVIII formulation has a pH from about pH 6.0 to about pH 6.5, or about pH 7.0, or about pH 7.5.

16. The BDD-rFVIII formulation of claim 15, comprising: about 20 mM MOPS, about 29 mM of a sugar or sugar alcohol, about 10 mM calcium chloride, about 220 mM sodium chloride, and about 100 ppm non-ionic surfactant.

17. A B-domain deleted rFVIII mutant (BDD-rFVIII-mutant) formulation, comprising:
a. from about 0 mM to about 20 mM or about 50 mM MOPS;
b. from about 0 mM to about 29 mM, or about 34 mM, or about 58 mM, or about 100 mM, or about 300 mM of a sugar or sugar alcohol;
c. from about 1 mM to about 2 mM, or about 2.5 mM, or about 5 mM, or about 10 mM, or about 15 mM calcium chloride;
d. from about 100 mM to about 150 mM, or about 200 mM, or about 220 mM, or about 250 mM sodium chloride;
e. from about 20 ppm to about 50 ppm, or about 80 ppm, or about 100 ppm, or about 120 ppm, or about 200 ppm of a non-ionic surfactant; and
f. from about 0.1 μg/ml to about 300 μg/ml, or from about 1.0 μg/ml to about 200 μg/ml, or from about 10 μg/ml to about 125 μg/ml of a BDD-rFVIII-mutant;
wherein said BDD-rFVIII-mutant formulation has a pH from about pH 6.0 to about pH 6.5, or about pH 7.0, or about pH 7.5.

18. The BDD-rFVIII-mutant formulation of claim 17, comprising: about 20 mM MOPS, about 29 mM of a sugar or sugar alcohol, about 10 mM calcium chloride, about 220 mM sodium chloride, and about 100 ppm non-ionic surfactant.

19. A PEGylated full-length rFVIII (PEG FL-rFVIII) formulation, comprising:
a. from about 0 mM to about 20 mM or about 50 mM MOPS;
b. from about 0 mM to about 29 mM, or about 34 mM, or about 58 mM, or about 100 mM, or about 300 mM of a sugar or sugar alcohol;
c. from about 1 mM to about 2 mM, or about 2.5 mM, or about 5 mM, or about 10 mM, or about 15 mM calcium chloride;
d. from about 100 mM to about 150 mM, or about 200 mM, or about 220 mM, or about 250 mM sodium chloride;
e. from about 20 ppm to about 50 ppm, or about 80 ppm, or about 100 ppm, or about 120 ppm, or about 200 ppm of a non-ionic surfactant; and
f. from about 0.1 μg/ml to about 300 μg/ml, or from about 1.0 μg/ml to about 200 μg/ml, or from about 10 μg/ml to about 125 μg/ml of a PEG FL-rFVIII;
wherein said PEG FL-rFVIII formulation has a pH from about pH 6.0 to about pH 6.5, or about pH 7.0, or about pH 7.5.

20. The PEG FL-rFVIII formulation of claim 19, comprising: about 20 mM MOPS, about 29 mM of a sugar or sugar alcohol, about 10 mM calcium chloride, about 220 mM sodium chloride, and about 100 ppm non-ionic surfactant.

21. A PEGylated B-domain deleted rFVIII (PEG-BDD-rFVIII) formulation, comprising:
a. from about 0 mM to about 20 mM or about 50 mM MOPS;
b. from about 0 mM to about 29 mM, or about 34 mM, or about 58 mM, or about 100 mM, or about 300 mM of a sugar or sugar alcohol;
c. from about 1 mM to about 2 mM, or about 2.5 mM, or about 5 mM, or about 10 mM, or about 15 mM calcium chloride;
d. from about 100 mM to about 150 mM, or about 200 mM, or about 220 mM, or about 250 mM sodium chloride;
e. from about 20 ppm to about 50 ppm, or about 80 ppm, or about 100 ppm, or about 120 ppm, or about 200 ppm of a non-ionic surfactant; and
f. from about 0.1 μg/ml to about 300 μg/ml, or from about 1.0 μg/ml to about 200 μg/ml, or from about 10 μg/ml to about 125 μg/ml of a PEG-BDD-rFVIII;
wherein said PEG-BDD-rFVIII formulation has a pH from about pH 6.0 to about pH 6.5, or about pH 7.0, or about pH 7.5.

22. The PEG-BDD-rFVIII formulation of claim 21, comprising: about 20 mM MOPS, about 29 mM of a sugar or sugar alcohol, about 10 mM calcium chloride, about 220 mM sodium chloride, and about 100 ppm non-ionic surfactant.

23. A PEGylated B-domain deleted rFVIII-mutant (PEG BDD-rFVIII-mutant) formulation, comprising:
a. from about 0 mM to about 20 mM or about 50 mM MOPS;
b. from about 0 mM to about 29 mM, or about 34 mM, or about 58 mM, or about 100 mM, or about 300 mM of a sugar or sugar alcohol;
c. from about 1 mM to about 2 mM, or about 2.5 mM, or about 5 mM, or about 10 mM, or about 15 mM calcium chloride;
d. from about 100 mM to about 150 mM, or about 200 mM, or about 220 mM, or about 250 mM sodium chloride;
e. from about 20 ppm to about 50 ppm, or about 80 ppm, or about 100 ppm, or about 120 ppm, or about 200 ppm of a non-ionic surfactant; and
f. from about 0.1 μg/ml to about 300 μg/ml, or from about 1.0 μg/ml to about 200 μg/ml, or from about 10 μg/ml to about 125 μg/ml of a PEG BDD-rFVIII-mutant;
wherein said PEG BDD-rFVIII-mutant formulation has a pH from about pH 6.0 to about pH 6.5, or about pH 7.0, or about pH 7.5.

24. The PEG BDD-rFVIII-mutant formulation of claim 23, comprising: about 20 mM MOPS, about 29 mM of a sugar or sugar alcohol, about 10 mM calcium chloride, about 220 mM sodium chloride, and about 100 ppm non-ionic surfactant.

25. A FL-rFVIII formulation, comprising:
a. from about 0 mM to about 20 mM or about 50 mM histidine;
b. from about 0 mM to about 29 mM, or about 34 mM, or about 58 mM, or about 100 mM, or about 300 mM of a sugar or sugar alcohol;
c. from about 1 mM to about 2 mM, to about 2.5 mM, to about 5 mM calcium chloride;
d. from about 0 mM to about 10 mM, or about 20 mM, or about 30 mM, or about 40 mM, or about 50 mM sodium chloride;
e. from about 20 ppm to about 50 ppm, or about 80 ppm, or about 100 ppm, or about 120 ppm, or about 200 ppm of a non-ionic surfactant;
f. from about 0 mM to about 50 mM, or about 100 mM, or about 150 mM, or about 293 mM, or about 400 mM glycine; and
g. from about 0.1 μg/ml to about 300 μg/ml, or about 1.0 μg/ml to about 200 μg/ml, or about 10 μg/ml to about 125 μg/ml of a FL-rFVIII;
wherein said FL-rFVIII formulation has a pH from about pH 6.0 to about pH 6.5, or about pH 7.0, or about pH 7.5.

26. The FL-rFVIII formulation of claim 25, comprising about 20 mM histidine, about 29 mM of a sugar or sugar alcohol, about 2.5 mM calcium chloride, about 30 mM sodium chloride, about 80 ppm of a non-ionic surfactant, about 293 mM glycine, and about 50 μg/ml FL-rFVIII; wherein said FL-rFVIII formulation has a pH from about pH 6.5 to about pH 7.0.

27. The FL-rFVIII formulation of claim 25, comprising about 20 mM histidine, about 37 mM of a sugar or sugar alcohol, about 2.5 mM calcium chloride, about 30 mM sodium chloride, about 80 ppm of a non-ionic surfactant, about 346 mM glycine, and about 50 μg/ml FL-rFVIII; wherein said FL-rFVIII formulation has a pH from about pH 6.5 to about pH 7.0.

28. The FL-rFVIII formulation of claim 25, comprising about 20 mM histidine, from about 100 mM to about 300 mM of a sugar or sugar alcohol, about 2.5 mM calcium chloride, about 0 mM sodium chloride, about 80 ppm of a non-ionic surfactant, about 0 mM glycine, and about 14 μg/ml FL-rFVIII; wherein said FL-rFVIII formulation has a pH from about pH 6.5 to about pH 7.0.

29. The FL-rFVIII formulation claim 25 wherein said sugar or sugar alcohol is selected from the group consisting of sucrose and trehalose.

30. The FL-rFVIII formulation claim 25 wherein said non-ionic surfactant is selected from the group consisting of polysorbate 20 and polysorbate 80.

31. A PEG-FL-rFVIII formulation, comprising:
a. from about 0 mM to about 20 mM or about 50 mM histidine;
b. from about 0 mM to about 29 mM, or about 34 mM, or about 58 mM, or about 100 mM, or about 300 mM of a sugar or sugar alcohol;
c. from about 1 mM to about 2 mM, to about 2.5 mM, to about 5 mM calcium chloride;
d. from about 0 mM to about 10 mM, or about 20 mM, or about 30 mM, or about 40 mM, or about 50 mM sodium chloride;
e. from about 20 ppm to about 50 ppm, or about 80 ppm, or about 100 ppm, or about 120 ppm, or about 200 ppm of a non-ionic surfactant;
f. from about 0 mM to about 50 mM, or about 100 mM, or about 150 mM, or about 293 mM, or about 400 mM glycine; and
g. from about 0.1 μg/ml to about 300 μg/ml, or about 1.0 μg/ml to about 200 μg/ml, or about 10 μg/ml to about 125 μg/ml of a PEG-FL-rFVIII;
wherein said PEG-FL-rFVIII formulation has a pH from about pH 6.0 to about pH 6.5, or about pH 7.0, or about pH 7.5.

32. The PEG-FL-rFVIII formulation of claim 31, comprising about 20 mM histidine, about 29 mM of a sugar or sugar alcohol, about 2.5 mM calcium chloride, about 30 mM sodium chloride, about 80 ppm of a non-ionic surfactant, about 293 mM glycine, and about 50 μg/ml PEG-FL-rFVIII; wherein said PEG-FL-rFVIII formulation has a pH from about pH 6.5 to about pH 7.0.

33. The PEG-FL-rFVIII formulation of claim 31, comprising about 20 mM histidine, about 37 mM of a sugar or sugar alcohol, about 2.5 mM calcium chloride, about 30 mM sodium chloride, about 80 ppm of a non-ionic surfactant, about 346 mM glycine, and about 50 μg/ml PEG-FL-rFVIII; wherein said PEG-FL-rFVIII formulation has a pH from about pH 6.5 to about pH 7.0.

34. The PEG-FL-rFVIII formulation of claim 31, comprising about 20 mM histidine, from about 100 mM to about 300 mM of a sugar or sugar alcohol, about 2.5 mM calcium chloride, about 0 mM sodium chloride, about 80 ppm of a non-ionic surfactant, about 0 mM glycine, and about 14 μg/ml PEG-FL-rFVIII; wherein said PEG-FL-rFVIII formulation has a pH from about pH 6.5 to about pH 7.0.

35. The PEG-FL-rFVIII formulation claim 31 wherein said sugar or sugar alcohol is selected from the group consisting of sucrose and trehalose.

36. The PEG-FL-rFVIII formulation claim 31 wherein said non-ionic surfactant is selected from the group consisting of polysorbate 20 and polysorbate 80.

37. A PEG-BDD-rFVIII formulation, comprising:
a. from about 0 mM to about 20 mM or about 50 mM histidine;
b. from about 0 mM to about 29 mM, or about 34 mM, or about 58 mM, or about 100 mM, or about 300 mM of a sugar or sugar alcohol;
c. from about 1 mM to about 2 mM, to about 2.5 mM, to about 5 mM calcium chloride;
d. from about 0 mM to about 10 mM, or about 20 mM, or about 30 mM, or about 40 mM, or about 50 mM sodium chloride;
e. from about 20 ppm to about 50 ppm, or about 80 ppm, or about 100 ppm, or about 120 ppm, or about 200 ppm of a non-ionic surfactant;
f. from about 0 mM to about 50 mM, or about 100 mM, or about 150 mM, or about 293 mM, or about 400 mM glycine; and
g. from about 0.1 μg/ml to about 300 μg/ml, or about 1.0 μg/ml to about 200 μg/ml, or about 10 μg/ml to about 125 μg/ml of a PEG-BDD-rFVIII;
wherein said PEG-BDD-rFVIII formulation has a pH from about pH 6.0 to about pH 6.5, or about pH 7.0, or about pH 7.5.

38. The PEG-BDD-rFVIII formulation of claim 37, comprising about 20 mM histidine, about 29 mM of a sugar or sugar alcohol, about 2.5 mM calcium chloride, about 30 mM sodium chloride, about 80 ppm of a non-ionic surfactant, about 293 mM glycine, and about 50 μg/ml PEG-BDD-rFVIII; wherein said PEG-BDD-rFVIII formulation has a pH from about pH 6.5 to about pH 7.0.

39. The PEG-BDD-rFVIII formulation of claim 37, comprising about 20 mM histidine, about 37 mM of a sugar or sugar alcohol, about 2.5 mM calcium chloride, about 30 mM sodium chloride, about 80 ppm of a non-ionic surfactant, about 346 mM glycine, and about 50 μg/ml PEG-BDD-rFVIII; wherein said PEG-BDD-rFVIII formulation has a pH from about pH 6.5 to about pH 7.0.

40. The PEG-BDD-rFVIII formulation of claim 37, comprising about 20 mM histidine, from about 100 mM to about 300 mM of a sugar or sugar alcohol, about 2.5 mM calcium chloride, about 0 mM sodium chloride, about 80 ppm of a non-ionic surfactant, about 0 mM glycine, and about 14 μg/ml PEG-BDD-rFVIII; wherein said PEG-BDD-rFVIII formulation has a pH from about pH 6.5 to about pH 7.0.

41. The PEG-BDD-rFVIII formulation claim 37 wherein said sugar or sugar alcohol is selected from the group consisting of sucrose and trehalose.

42. The PEG-BDD-rFVIII formulation claim 37 wherein said non-ionic surfactant is selected from the group consisting of polysorbate 20 and polysorbate 80.

43. A PEG BDD-rFVIII-mutant formulation, comprising:
a. from about 0 mM to about 20 mM or about 50 mM histidine;
b. from about 0 mM to about 29 mM, or about 34 mM, or about 58 mM, or about 100 mM, or about 300 mM of a sugar or sugar alcohol;
c. from about 1 mM to about 2 mM, to about 2.5 mM, to about 5 mM calcium chloride;
d. from about 0 mM to about 10 mM, or about 20 mM, or about 30 mM, or about 40 mM, or about 50 mM sodium chloride;
e. from about 20 ppm to about 50 ppm, or about 80 ppm, or about 100 ppm, or about 120 ppm, or about 200 ppm of a non-ionic surfactant;
f. from about 0 mM to about 50 mM, or about 100 mM, or about 150 mM, or about 293 mM, or about 400 mM glycine; and
g. from about 0.1 μg/ml to about 300 μg/ml, or about 1.0 μg/ml to about 200 μg/ml, or about 10 μg/ml to about 125 μg/ml of a PEG BDD-rFVIII-mutant;
wherein said PEG BDD-rFVIII-mutant formulation has a pH from about pH 6.0 to about pH 6.5, or about pH 7.0, or about pH 7.5.

44. The PEG BDD-rFVIII-mutant formulation of claim 43, comprising about 20 mM histidine, about 29 mM of a sugar or sugar alcohol, about 2.5 mM calcium chloride, about 30 mM sodium chloride, about 80 ppm of a non-ionic surfactant, about 293 mM glycine, and about 50 μg/ml PEG BDD-rFVIII-mutant; wherein said PEG BDD-rFVIII-mutant formulation has a pH from about pH 6.5 to about pH 7.0.

45. The PEG BDD-rFVIII-mutant formulation of claim 43, comprising about 20 mM histidine, about 37 mM of a sugar or sugar alcohol, about 2.5 mM calcium chloride, about 30 mM sodium chloride, about 80 ppm of a non-ionic surfactant, about 346 mM glycine, and about 50 μg/ml PEG BDD-rFVIII-mutant; wherein said PEG BDD-rFVIII-mutant formulation has a pH from about pH 6.5 to about pH 7.0.

46. The PEG BDD-rFVIII-mutant formulation of claim 43, comprising about 20 mM histidine, from about 100 mM to about 300 mM of a sugar or sugar alcohol, about 2.5 mM calcium chloride, about 0 mM sodium chloride, about 80 ppm of a non-ionic surfactant, about 0 mM glycine, and about 14 μg/ml PEG BDD-rFVIII-mutant; wherein said PEG BDD-rFVIII-mutant formulation has a pH from about pH 6.5 to about pH 7.0.

47. The PEG BDD-rFVIII-mutant formulation claim 43 wherein said sugar or sugar alcohol is selected from the group consisting of sucrose and trehalose.

48. The PEG BDD-rFVIII-mutant formulation claim 43 wherein said non-ionic surfactant is selected from the group consisting of polysorbate 20 and polysorbate 80.

49. A FL-rFVIII formulation, comprising:
a. from about 0 mM to about 20 mM or about 50 mM MOPS;
b. from about 0 mM to about 29 mM, or about 34 mM, or about 58 mM, or about 100 mM, or about 300 mM of a sugar or sugar alcohol;
c. from about 1 mM to about 2 mM, to about 2.5 mM, to about 5 mM calcium chloride;
d. from about 0 mM to about 10 mM, or about 20 mM, or about 30 mM, or about 40 mM, or about 50 mM sodium chloride;
e. from about 20 ppm to about 50 ppm, or about 80 ppm, or about 100 ppm, or about 120 ppm, or about 200 ppm of a non-ionic surfactant;
f. from about 0 mM to about 50 mM, or about 100 mM, or about 150 mM, or about 293 mM, or about 400 mM glycine; and
g. from about 0.1 μg/ml to about 300 μg/ml, or about 1.0 μg/ml to about 200 μg/ml, or about 10 μg/ml to about 125 μg/ml of a FL-rFVIII;
wherein said FL-rFVIII formulation has a pH from about pH 6.0 to about pH 6.5, or about pH 7.0, or about pH 7.5.

50. The FL-rFVIII formulation of claim 49, comprising about 20 mM MOPS, about 29 mM of a sugar or sugar alcohol, about 2.5 mM calcium chloride, about 30 mM sodium chloride, about 80 ppm of a non-ionic surfactant, about 293 mM glycine, and about 50 μg/ml FL-rFVIII; wherein said FL-rFVIII formulation has a pH from about pH 6.5 to about pH 7.0.

51. The FL-rFVIII formulation of claim 49, comprising about 20 mM MOPS, about 37 mM of a sugar or sugar alcohol, about 2.5 mM calcium chloride, about 30 mM sodium chloride, about 80 ppm of a non-ionic surfactant, about 346 mM glycine, and about 50 μg/ml FL-rFVIII; wherein said FL-rFVIII formulation has a pH from about pH 6.5 to about pH 7.0.

52. The FL-rFVIII formulation of claim 49, comprising about 20 mM MOPS, from about 100 mM to about 300 mM of a sugar or sugar alcohol, about 2.5 mM calcium chloride, about 0 mM sodium chloride, about 80 ppm of a non-ionic surfactant, about 0 mM glycine, and about 14 μg/ml FL-rFVIII; wherein said FL-rFVIII formulation has a pH from about pH 6.5 to about pH 7.0.

53. The FL-rFVIII formulation claim 49 wherein said sugar or sugar alcohol is selected from the group consisting of sucrose and trehalose.

54. The FL-rFVIII formulation claim 49 wherein said non-ionic surfactant is selected from the group consisting of polysorbate 20 and polysorbate 80.

55. A PEG-FL-rFVIII formulation, comprising:
a. from about 0 mM to about 20 mM or about 50 mM MOPS;
b. from about 0 mM to about 29 mM, or about 34 mM, or about 58 mM, or about 100 mM, or about 300 mM of a sugar or sugar alcohol;
c. from about 1 mM to about 2 mM, to about 2.5 mM, to about 5 mM calcium chloride;
d. from about 0 mM to about 10 mM, or about 20 mM, or about 30 mM, or about 40 mM, or about 50 mM sodium chloride;
e. from about 20 ppm to about 50 ppm, or about 80 ppm, or about 100 ppm, or about 120 ppm, or about 200 ppm of a non-ionic surfactant;
f. from about 0 mM to about 50 mM, or about 100 mM, or about 150 mM, or about 293 mM, or about 400 mM glycine; and
g. from about 0.1 μg/ml to about 300 μg/ml, or about 1.0 μg/ml to about 200 μg/ml, or about 10 μg/ml to about 125 μg/ml of a PEG-FL-rFVIII;
wherein said PEG-FL-rFVIII formulation has a pH from about pH 6.0 to about pH 6.5, or about pH 7.0, or about pH 7.5.

56. The PEG-FL-rFVIII formulation of claim 55, comprising about 20 mM MOPS, about 29 mM of a sugar or sugar alcohol, about 2.5 mM calcium chloride, about 30 mM sodium chloride, about 80 ppm of a non-ionic surfactant, about 293 mM glycine, and about 50 μg/ml PEG-FL-rFVIII; wherein said PEG-FL-rFVIII formulation has a pH from about pH 6.5 to about pH 7.0.

57. The PEG-FL-rFVIII formulation of claim 55, comprising about 20 mM MOPS, about 37 mM of a sugar or sugar alcohol, about 2.5 mM calcium chloride, about 30 mM sodium chloride, about 80 ppm of a non-ionic surfactant, about 346 mM glycine, and about 50 μg/ml PEG-FL-rFVIII; wherein said PEG-FL-rFVIII formulation has a pH from about pH 6.5 to about pH 7.0.

58. The PEG-FL-rFVIII formulation of claim 55, comprising about 20 mM MOPS, from about 100 mM to about 300 mM of a sugar or sugar alcohol, about 2.5 mM calcium chloride, about 0 mM sodium chloride, about 80 ppm of a non-ionic surfactant, about 0 mM glycine, and about 14 μg/ml PEG-FL-rFVIII; wherein said PEG-FL-rFVIII formulation has a pH from about pH 6.5 to about pH 7.0.

59. The PEG-FL-rFVIII formulation claim 55 wherein said sugar or sugar alcohol is selected from the group consisting of sucrose and trehalose.

60. The PEG-FL-rFVIII formulation claim 55 wherein said non-ionic surfactant is selected from the group consisting of polysorbate 20 and polysorbate 80.

61. A PEG-BDD-rFVIII formulation, comprising:
a. from about 0 mM to about 20 mM or about 50 mM MOPS;
b. from about 0 mM to about 29 mM, or about 34 mM, or about 58 mM, or about 100 mM, or about 300 mM of a sugar or sugar alcohol;
c. from about 1 mM to about 2 mM, to about 2.5 mM, to about 5 mM calcium chloride;
d. from about 0 mM to about 10 mM, or about 20 mM, or about 30 mM, or about 40 mM, or about 50 mM sodium chloride;
e. from about 20 ppm to about 50 ppm, or about 80 ppm, or about 100 ppm, or about 120 ppm, or about 200 ppm of a non-ionic surfactant;
f. from about 0 mM to about 50 mM, or about 100 mM, or about 150 mM, or about 293 mM, or about 400 mM glycine; and
g. from about 0.1 μg/ml to about 300 μg/ml, or about 1.0 μg/ml to about 200 μg/ml, or about 10 μg/ml to about 125 μg/ml of a PEG-BDD-rFVIII;
wherein said PEG-BDD-rFVIII formulation has a pH from about pH 6.0 to about pH 6.5, or about pH 7.0, or about pH 7.5.

62. The PEG-BDD-rFVIII formulation of claim 61, comprising about 20 mM MOPS, about 29 mM of a sugar or sugar alcohol, about 2.5 mM calcium chloride, about 30 mM sodium chloride, about 80 ppm of a non-ionic surfactant, about 293 mM glycine, and about 50 μg/ml PEG-BDD-rFVIII; wherein said PEG-BDD-rFVIII formulation has a pH from about pH 6.5 to about pH 7.0.

63. The PEG-BDD-rFVIII formulation of claim 61, comprising about 20 mM MOPS, about 37 mM of a sugar or sugar alcohol, about 2.5 mM calcium chloride, about 30 mM sodium chloride, about 80 ppm of a non-ionic surfactant, about 346 mM glycine, and about 50 μg/ml PEG-BDD-rFVIII; wherein said PEG-BDD-rFVIII formulation has a pH from about pH 6.5 to about pH 7.0.

64. The PEG-BDD-rFVIII formulation of claim 61, comprising about 20 mM MOPS, from about 100 mM to about 300 mM of a sugar or sugar alcohol, about 2.5 mM calcium chloride, about 0 mM sodium chloride, about 80 ppm of a non-ionic surfactant, about 0 mM glycine, and about 14 μg/ml PEG-BDD-rFVIII; wherein said PEG-BDD-rFVIII formulation has a pH from about pH 6.5 to about pH 7.0.

65. The PEG-BDD-rFVIII formulation claim 61 wherein said sugar or sugar alcohol is selected from the group consisting of sucrose and trehalose.

66. The PEG-BDD-rFVIII formulation claim 61 wherein said non-ionic surfactant is selected from the group consisting of polysorbate 20 and polysorbate 80.

67. A PEG BDD-rFVIII-mutant formulation, comprising:
a. from about 0 mM to about 20 mM or about 50 mM MOPS;
b. from about 0 mM to about 29 mM, or about 34 mM, or about 58 mM, or about 100 mM, or about 300 mM of a sugar or sugar alcohol;
c. from about 1 mM to about 2 mM, to about 2.5 mM, to about 5 mM calcium chloride;
d. from about 0 mM to about 10 mM, or about 20 mM, or about 30 mM, or about 40 mM, or about 50 mM sodium chloride;
e. from about 20 ppm to about 50 ppm, or about 80 ppm, or about 100 ppm, or about 120 ppm, or about 200 ppm of a non-ionic surfactant;
f. from about 0 mM to about 50 mM, or about 100 mM, or about 150 mM, or about 293 mM, or about 400 mM glycine; and
g. from about 0.1 μg/ml to about 300 μg/ml, or about 1.0 μg/ml to about 200 μg/ml, or about 10 μg/ml to about 125 μg/ml of a PEG BDD-rFVIII-mutant;
wherein said PEG BDD-rFVIII-mutant formulation has a pH from about pH 6.0 to about pH 6.5, or about pH 7.0, or about pH 7.5.

68. The PEG BDD-rFVIII-mutant formulation of claim 67, comprising about 20 mM MOPS, about 29 mM of a sugar or sugar alcohol, about 2.5 mM calcium chloride, about 30 mM sodium chloride, about 80 ppm of a non-ionic surfactant, about 293 mM glycine, and about 50 μg/ml PEG BDD-rFVIII-mutant; wherein said PEG BDD-rFVIII-mutant formulation has a pH from about pH 6.5 to about pH 7.0.

69. The PEG BDD-rFVIII-mutant formulation of claim 67, comprising about 20 mM MOPS, about 37 mM of a sugar or sugar alcohol, about 2.5 mM calcium chloride, about 30 mM sodium chloride, about 80 ppm of a non-ionic surfactant, about 346 mM glycine, and about 50 μg/ml PEG BDD-rFVIII-mutant; wherein said PEG BDD-rFVIII-mutant formulation has a pH from about pH 6.5 to about pH 7.0.

70. The PEG BDD-rFVIII-mutant formulation of claim 67, comprising about 20 mM MOPS, from about 100 mM to about 300 mM of a sugar or sugar alcohol, about 2.5 mM calcium chloride, about 0 mM sodium chloride, about 80 ppm of a non-ionic surfactant, about 0 mM glycine, and about 14 μg/ml PEG BDD-rFVIII-mutant; wherein said PEG BDD-rFVIII-mutant formulation has a pH from about pH 6.5 to about pH 7.0.

71. The PEG BDD-rFVIII-mutant formulation claim 67 wherein said sugar or sugar alcohol is selected from the group consisting of sucrose and trehalose.

72. The PEG BDD-rFVIII-mutant formulation claim 67 wherein said non-ionic surfactant is selected from the group consisting of polysorbate 20 and polysorbate 80.

73. A method for the treatment of hemophilia A in a patient, said method comprising administering to said patient a therapeutically effective amount of a formulation claim 1.

74. The method of claim 73 wherein said formulation is administered intravenously, subcutaneously, or by continuous infusion.
